(19) United States
(12) Patent Application Publication
(10) Pub. No.: US 2026/0284101 A1
(43) Pub. Date: Sep. 24, 2026

(54) IRRITATION RELIEVING AGENT, METHOD FOR PRODUCING THE SAME, AND EXTERNAL SKIN PREPARATION

(71) Applicant: REFINE HOLDINGS CO., LTD., Gifu (JP)

(72) Inventors: Makoto TSUBOI, Tokyo (JP); Shogo HAMAMOTO, Tokyo (JP)

(73) Assignee: REFINE HOLDINGS CO., LTD., Gifu (JP)

(21) Appl. No.: 19/477,880

(22) PCT Filed: May 8, 2024
(86) PCT No.: PCT/JP2024/017103

§ 371 Date: Oct. 23, 2025

(30) Foreign Application Priority Data
May 9, 2023 (JP) 2023-077229

(51) Int. Cl. A61K 31/737; A61K 9/00; A61K 36/04; A61P 17/04

(52) U.S. Cl. CPC A61K 31/737; A61K 9/0014; A61K 36/04; A61P 17/04; A61K 2236/331; A61K 2236/39

(57) ABSTRACT

To provide an irritation relieving agent that has high safety and excellent irritation suppressing ability. The irritation relieving agent is characterized by comprising a sulfated polysaccharide salt that has a sulfate group in at least one saccharide unit structure, and in which the sulfate group forms a salt with a monovalent cation.

### TECHNICAL FIELD

The present invention relates to an irritation relieving agent that is added to and used in compositions that come into contact with the skin, such as cosmetics and pharmaceuticals, a method for producing the same, and an external skin preparation.

### BACKGROUND ART

Since safety is considered highly important for cosmetic ingredients, they are used within a concentration range that causes almost no irritation to the skin. However, when the skin's barrier function is compromised, even ingredients that generally cause little skin irritation may cause skin irritation. To alleviate irritation to the skin, ingredients that repair the skin barrier function or ingredients that relieve the irritation caused by other components have been reported. For example, Patent Literature 1 discloses that a composition containing a carbohydrate or a derivative thereof as an active ingredient relieves skin irritation caused by a dye and prevents or inhibits inflammation caused by a dye. Specific examples of the carbohydrate or its derivative include one or more selected from the group consisting of monosaccharides, oligosaccharides, and polysaccharides.

Patent Literature 2 also discloses that a polysaccharide (levan) produced by the action of a microorganism-derived enzyme has an effect of alleviating skin irritation. Levan is a fructose polymer linked by β(2→6) bonds and is produced from sucrose as a substrate by various microorganisms such as Bacillus subtilis. However, until now, there has been no naturally derived irritation relieving agent that sufficiently suppresses the irritation caused by lactic acid, which is a typical irritant in cosmetics, and that is excellent in its own safety and stability.

### PRIOR ART DOCUMENTS

### Patent Literature

Patent Literature 1: JP 2013-511512 A
Patent Literature 2: JP 2003-277225 A

### SUMMARY OF THE INVENTION

### Problems to be Solved by the Invention

The present invention has been made in view of such circumstances, and an object of the present invention is to provide an irritation relieving agent that has high safety and excellent irritation suppressing ability.

### Means for Solving the Problems

The present inventors conducted intensive studies to solve the above problem and, as a result, found that a specific polysaccharide molecule, which exhibits excellent functionality in a Funori polysaccharide extracted from Funori by a specific extraction method, can alleviate irritation to the skin caused by pharmaceuticals and cosmetics when added to an external skin preparation. That is, the present invention includes the following embodiments.
(1) An irritation relieving agent comprising a sulfated polysaccharide salt having a sulfate group in at least one saccharide unit structure, wherein the sulfate group forms a salt with a monovalent cation.
(2) The irritation relieving agent according to (1), wherein the sulfated polysaccharide salt has one or more hexose constituent units selected from the group consisting of fucose, rhamnose, galactose, glucose, mannose, and fructose, and has a sulfate group in at least one of these hexose constituent units.
(3) The irritation relieving agent according to (1), wherein the sulfated polysaccharide salt is funoran sodium salt, which is a polysaccharide derived from Funori.
(4) An external skin preparation comprising the irritation relieving agent according to any one of (1) to (3) in an amount of 0.000001% by mass to 0.01% by mass in terms of solid content of the sulfated polysaccharide salt.
(5) A method for producing an irritation relieving agent, comprising the steps of: dissolving Funori powder in water to prepare an aqueous Funori solution; adding a monovalent cation to the aqueous Funori solution and heating; and fractionating the aqueous solution after heating by molecular weight fractionation to recover the sulfated polysaccharide salt.
(6) A method for producing an irritation relieving agent, comprising the steps of: dissolving Funori powder in water to prepare an aqueous Funori solution; adding an acid to the aqueous Funori solution to make it acidic, followed by desalting; adding a monovalent cation to the aqueous solution after desalting to neutralize it; and fractionating the aqueous solution after neutralization by molecular weight fractionation to recover the sulfated polysaccharide salt.

### Effects of the Invention

The irritation relieving agent according to the present invention can alleviate skin irritation caused by pharmaceuticals and cosmetics even with a very small amount of use.

### BRIEF DESCRIPTION OF THE DRAWINGS

FIG. 1 shows the results of the irritation relieving test of funoran Na conducted in Example 1.

### MODE FOR CARRYING OUT THE INVENTION

Hereinafter, the irritation relieving agent, the method for producing the same, and the external skin preparation containing the irritation relieving agent according to the embodiments of the present invention will be described in detail, but not all of the elements and combinations thereof described in each embodiment are essential for the means for solving the problem of the present invention.

### (Irritation Relieving Agent)

The irritation relieving agent according to this embodiment is characterized by comprising a sulfated polysaccharide salt as an active ingredient, which has a sulfate group in at least one saccharide unit structure, and in which the sulfate group forms a salt with a monovalent cation. The structure of the polysaccharide constituting this sulfated polysaccharide salt is not particularly limited as long as it has a sulfate group in at least one saccharide unit structure. The saccharide unit structure preferably includes a hexose, and may be a homopolysaccharide composed of one type of hexose constituent unit, or a heteropolysaccharide containing at least one type of hexose constituent unit and composed of two or more types of saccharide constituent units.

In the case of a heteropolysaccharide, the saccharide constituent units other than the at least one type of hexose constituent unit may be a triose constituent unit, a tetrose constituent unit, a pentose constituent unit, or a hexose constituent unit, and two or more saccharide constituent units can be constituted by any combination of these.

The hexose constituent units contained in the polysaccharide constituting the sulfated polysaccharide salt according to this embodiment are not particularly limited, and include, for example, fucose, rhamnose, galactose, glucose, mannose, and fructose, or those having one or more of these hexose constituent units. It is sufficient that at least a part of the hexose constituent units has a sulfate group.

The sulfated polysaccharide salt according to this embodiment may have a sulfate group at any position in the saccharide unit structure of the polysaccharide constituting it. For example, it may have a sulfate group at the 2, 3, 4, 5, and/or 6-position of the hexose constituent unit, but it is preferably a sulfated polysaccharide salt having a sulfate group at the 4 and/or 6-position, and most preferably one having a sulfate group at the 6-position. Examples include those in which the OH group at the 4-position of fucose or the OH group at the 6-position of galactose is sulfated (half-sulfate ester).

Furthermore, the sulfated polysaccharide salt according to this embodiment is not particularly limited as long as at least a part of the hexose constituent units in the saccharide unit structure of the polysaccharide constituting it has a sulfate group. It is desirable that the constituent unit having a sulfate group is contained, for example, at a ratio of 0.1 to 100%, preferably 1 to 100%, 10 to 100%, 30 to 100%, 40 to 100%, or 50 to 100% of the total saccharide unit structures constituting the sulfated polysaccharide salt. Moreover, if 50% or more, preferably 60% or more, 70% or more, 80% or more, 90% or more, or 95% or more of the total saccharide unit structures have a hexose constituent unit with a sulfate group, the effect is substantially the same as that of a compound in which 100% of the total saccharide unit structures have a saccharide unit structure with a sulfate group.

The sulfated polysaccharide salt according to this embodiment is further salified with a monovalent cation, such as Na⁺, K⁺, or NH₄⁺, preferably Na⁺ and K⁺, and particularly preferably Na⁺. That is, it was revealed that the above-described polysaccharide having a sulfate group cannot exhibit its predetermined function unless it is in a salt state because self-hydrolysis progresses, and when the counter ion forming the salt is a divalent cation such as Ca²⁺, the irritation relieving function, such as alleviating irritation to the skin caused by pharmaceuticals and cosmetics, is not observed, even in the sulfated polysaccharide salt having a sulfate group in the saccharide unit structure. Such an irritation relieving function is only achieved when it is a salt of a monovalent cation. Therefore, it is preferable that the sulfated polysaccharide salt according to the present invention substantially does not contain a divalent cation such as calcium. Here, “substantially does not contain” means, for example, that the amount present is 5% or less, 1% or less, 0.1% or less, 0.01% or less, or 0.001% or less in molar units, and more preferably an amount close to zero or less.

The sulfated polysaccharide salt according to this embodiment may be one obtained by synthesis or one extracted from various natural raw materials, as long as it has the above-described structure, but a naturally derived one is preferable, and one derived from Funori, such as funoran sodium salt, is particularly desirable. Funori is a seaweed belonging to the genus Gloiopeltis (scientific name), family Funoriaceae, class Rhodophyceae, and Gloiopeltis furcata (Fukurofnori), Gloiopeltis tenax (Mafnori), and Gloiopeltis complanata (Hanafnori) are well known in Japan. The Funori used in the present invention may be the one from which foreign substances have been removed, appropriately purified, dried, and sheared or pulverized, or it may be one from which an extract has been obtained once and then granulated or pulverized.

Although not particularly limited, the sulfated polysaccharide salt related to funoran sodium salt is exemplified by those having a structural formula such as the following.

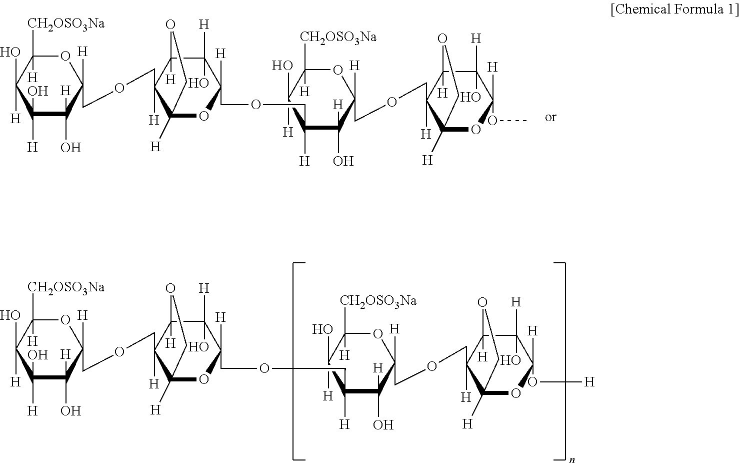

(where n is 1-20000, more preferably, for example, 1-10000, 10-8000, 100-5000, 200-4000, or 1000-2000.)

The irritation relieving agent according to this embodiment contains the above-described sulfated polysaccharide salt, and its content can be adjusted to a desired concentration from 0.00001% by mass to 99% by mass in terms of solid content of the sulfated polysaccharide salt. The irritation relieving agent of this embodiment is an ingredient that alleviates the irritation of other components, and it is preferably used as an additive for manufacturing pharmaceuticals or cosmetics containing desired active ingredients.

### (External Skin Preparation)

The external skin preparation according to this embodiment contains the above-described irritation relieving agent.

Examples of the external skin preparation of this embodiment include: basic cosmetics such as skin lotion, milky lotion, cream, ointment, lotion, oil, and pack; skin cleansing products such as soap, cleansing cream, cleansing lotion, and face wash; hair washing cosmetics such as shampoo, rinse, and treatment; hair styling products such as hair cream, hair spray, hair tonic, hair gel, hair lotion, hair oil, hair essence, hair water, hair wax, and hair foam; hair growth/nourishment agents; make-up cosmetics such as foundation, face powder, loose powder, lipstick, cheek blush, eyeshadow, eyeliner, mascara, eyebrow pencil, and eyelash products; finishing cosmetics such as nail care products; cosmetic compositions such as perfumes, toothpastes, and oral compositions such as mouthwashes; external medicinal preparations, ointments, poultices, bath agents; medicated oral compositions such as medicated toothpastes and mouth refreshers; medicated cosmetics, hair dyes, hair growth agents, hair loss preventives, hair removal agents; body odor/antiperspirant agents for hair solvent liquids; external quasi-drugs such as sanitary goods, sanitary cotton, and wet wipes; and external drugs.

The content of the irritation relieving agent in the external skin preparation according to this embodiment is not particularly limited. For example, in a composition in the form of an aqueous solution, a very small amount of the sulfated polysaccharide salt in terms of solid content, such as 0.000001% by mass or more, preferably 0.00001% by mass or more, and more preferably 0.0001% by mass or more of the total external skin preparation, can exhibit a very good skin irritation alleviating effect. The upper limit of the content is also not particularly limited, but from an economic point of view, it is desirable that it is, for example, 0.1% by mass or less, particularly 0.05% by mass, or even 0.01% by mass or less. A particularly preferable range is 0.00005% by mass to 0.0005% by mass.

In addition to the above-described ingredients, the external skin preparation may appropriately contain the following substances depending on the required efficacy and effects. Examples include various cosmetic ingredients such as oils and fats, surfactants, humectants, whitening agents, pH adjusters, binding agents and thickening agents, polyhydric alcohols, essential oils and fragrances, preservatives, antioxidants, UV absorbers, pigments, pulverized plant materials and crude drugs, inorganic salts and inorganic acids, cleansing agents, and emulsifiers.

Examples of the oils and fats include those commonly used in cosmetics, such as: vegetable oils and fats like soybean oil, almond oil, paraffin, cetanol, avocado oil, olive oil, jojoba oil, coconut oil, palm oil, rice bran oil, egg yolk oil, castor oil, squalane, liquid paraffin, and white petrolatum; animal oils and fats like beef tallow, lard, horse fat, turtle oil, mink oil, perhydrosqualene, and squalane; and synthetic oils and fats like methylpolysiloxane, behenyl alcohol, glyceryl tricaprylate, glyceryl trioctanoate, liquid paraffin, DHA, and EPA.

Examples of the surfactants include: anionic surfactants such as sodium lauryl sulfate, triethanol amine lauryl sulfate, and diethanol amide laurate; cationic surfactants such as stearyl trimethyl ammonium chloride, cetyl trimethyl ammonium chloride, and benzalkonium chloride; nonionic surfactants such as glyceryl monostearate, sorbitan monostearate, polyoxyethylene (20) sorbitan monostearate, polyoxyethylene hydrogenated castor oil, sucrose ester, and fatty acid amide; and polymer surfactants, fluorine-based surfactants, silicone-based surfactants, polypeptide derivatives, and natural surfactants.

Examples of the humectants include: synthetic humectants such as sodium pyrrolidonecarboxylate and pantetheine-S-sulfonate; and natural humectants such as hyaluronic acid, elastin, keratin, dermatan sulfate, collagen, placental extract, royal jelly, and their hydrolysates, microbial fermentation liquids (e.g., chitin, chitosan, pectin, glucosamine, N-acetyl glucosamine), other plant and animal derived extracts, vegetable oils and their esterified products and derivatives.

Examples of the whitening agents include ascorbic acid and its salts and derivatives, arbutin and its derivatives, placental extract, and other plant and animal derived extracts.

Examples of the pH adjusters include organic acids, inorganic acids, and their salts such as citric acid, lactic acid, salicylic acid, tartaric acid, malic acid, benzoic acid, sodium citrate, fumaric acid, disodium hydrogen phosphate, and potassium dihydrogen phosphate.

Examples of the binding agents and thickening agents include those commonly used in cosmetics, such as sodium carboxymethylcellulose, methylcellulose, casein, pectin, starch, sodium alginate, polyvinyl alcohol, polyvinylpyrrolidone, locust bean gum, agar, carbomer, xanthan gum, and bentonite.

Examples of the polyhydric alcohols include glycerin, propylene glycol, sorbitol, polyethylene glycol, 1,3-butylene glycol, dipropylene glycol, and polyoxyethylene glycerin (26E. O).

Examples of the essential oils and fragrances include natural and synthetic fragrances such as lavender oil, jasmine oil, lemon oil, orange oil, peppermint oil, thyme oil, calamus oil, fennel oil, cedar oil, Thujopsis dolabrata oil, raspberry oil, cypress oil, rose oil, eucalyptus oil, camphor, spearmint oil, geraniol, mandarin oil, spruce oil, citronellol, terpenes, coffee oil, tea oil, camellia oil, and raspberry ketone.

Examples of the preservatives include parahydroxy benzoic acid esters such as methyl paraben, ethyl paraben, propyl paraben, and butyl paraben, phenoxy ethanol, ethanol, and dehydroacetic acid.

Examples of the antioxidants include EDTA4Na, EDTA2Na, butylhydroxytoluene, hydroxybutyltoluene, butylated hydroxyanisole (BHA), dibutylhydroxytoluene (BHT), and their derivatives.

The UV absorbers are not particularly limited as long as they have the property of selectively absorbing ultraviolet rays, and examples include oxybenzone, oxybenzone sulfonic acid, tetrahydroxy benzophenone, sodium dihydroxy-methoxy benzophenone sulfonate, dihydroxy-methoxy benzophenone, dihydroxy benzophenone, cinoxate, methyl diisopropyl cinnamate, octyl methoxy cinnamate, glyceryl para-aminobenzoate, and octyl para-aminobenzoate.

Examples of the pigments include red iron oxide, yellow iron oxide, black iron oxide, titanium oxide, nylon powder, zinc oxide, sericite, mica, talc, carbon, and medicinal charcoal.

Examples of the pulverized plant materials and crude drugs include pulverized materials, dried products, and extracts of lemon peel, seaweed, Hinoki cypress, Hiba cedar, Fucus, rice bran, calamus, ginger, licorice, citrus peel, yuzu, Japanese angelica root, carrot, mint, cinnamon, Ubai (unripe apricot), mugwort, Dokudami (Houttuynia cordata), peach leaf, chamomile, aloe, jasmine, rose hip, lavender, guava, Ougon (Scutellaria root), goji berry, reishi mushroom, elderberry, Ashitaba (Angelica keiskei), Ukogi (Acanthopanax), burdock, black sesame, black rice, Fuji Sanchin ginseng, Korean ginseng, Tienchi ginseng, Sekkotsu-so (Lycopodium clavatum), and Senkyu (Cnidium rhizome).

Examples of the inorganic salts and inorganic acids include sodium chloride, sodium bicarbonate, sodium carbonate, boric acid, borax, sodium sulfate, sodium sulfide, potassium sulfide, sodium nitrate, calcium nitrate, ammonium sulfate, sodium thiosulfate, calcium hydrogen phosphate, potassium chloride, ammonium chloride, sodium phosphate, sodium hydrosulfite, calcium thiosulfate, sulfur, sodium sesquicarbonate, magnesium sulfate, magnesium chloride, anhydrous silicic acid, metasilicic acid, magnesium carbonate, magnesium hydroxide, magnesium dihydrogen, and dried seawater.

Examples of the cleansing agents include anionic surfactants such as fatty acid soap, sodium lauryl sulfate, and alkyl ether carboxylate, and aliphatic compounds.

As the emulsifiers, substances known as cosmetic raw materials can be used. Particularly preferred examples include: sucrose fatty acid esters such as sucrose laurate, sucrose myristate, sucrose palmitate, sucrose stearate, sucrose oleate, and sucrose erucate; polyglycerin fatty acid esters such as tetraglyceryl monostearate, tetraglyceryl monooleate, tetraglyceryl tristearate, hexaglyceryl monolaurate, hexaglyceryl monomyristate, hexaglyceryl monostearate, hexaglyceryl tristearate, decaglyceryl monolaurate, decaglyceryl monomyristate, decaglyceryl monostearate, decaglyceryl distearate, decaglyceryl tristearate, decaglyceryl pentastearate, decaglyceryl monoisostearate, decaglyceryl diisostearate, decaglyceryl pentaisostearate, decaglyceryl monooleate, decaglyceryl trioleate, and decaglyceryl pentaoleate; sorbitan branched fatty acid esters such as sorbitan monoisostearate, sorbitan sesquiisostearate, and sorbitan triisostearate; lecithins such as soybean lecithin, egg yolk lecithin, hydrogenated soybean lecithin, hydrogenated egg yolk lecithin, lysolecithin which is the monoacyl form of these lecithins by enzyme treatment and/or hydrogenated lysolecithin, and hydroxylated hydroxy-lecithin; and glycerin branched fatty acid esters such as glyceryl monoisostearate, glyceryl sesquiisostearate, and glyceryl diisostearate. One or more of these can be used in combination.

Furthermore, the external skin preparation may appropriately contain other components as needed, in addition to the above, such as milk-derived components like lactose, milk, and condensed milk; inorganic pigments like titanium and talc; bactericides like isopropylmethylphenol, chlorhexidine hydrochloride, and chlorhexidine gluconate; vitamins and coenzymes like vitamin A, vitamin B1, vitamin B2, vitamin B6, vitamin P, CoQ10, CoQ9, CoQ8, and thioctic acid; fluidizing agents like anhydrous silicic acid and synthetic aluminum silicate; and pharmaceuticals, quasi-drugs, and tar-based dyes for cosmetics.

The external skin preparation may also appropriately contain various cosmetic ingredients, such as medicinal components for the purpose of beauty, facial care, and skin treatment, in addition to the above-mentioned components. Examples of the medicinal components include: avocado extract, Amacha extract (Hydrangea macrophylla var. thunbergii extract), Althaea extract, Arnica extract, apricot extract, apricot kernel extract, ginkgo extract, fennel extract, turmeric extract, oolong tea extract, Echinacea leaf extract, Oubaku extract (Phellodendron bark extract), barley extract, watercress extract, orange extract, hydrolyzed wheat powder, hydrolyzed silk, chamomile extract, carrot extract, Kawarayomogi extract (Artemisia capillaris extract), Karkade extract (hibiscus extract), kiwi extract, cucumber extract, guanosine, Kumazasa extract (Sasa veitchii extract), walnut extract, grapefruit extract, Clematis extract, yeast extract, comfrey extract, cranberry extract, Saiko extract (Bupleurum root extract), umbilical cord extract, sage extract, soapwort extract, Sasa extract (Sasa bamboo extract), hawthorn extract, shiitake mushroom extract, Jiou extract (Rehmannia root extract), Shikon extract (Lithospermum root extract), linden extract, meadowsweet extract, Shoubu root extract (Acorus root extract), birch extract, horsetail extract, honeysuckle extract, ivy extract, hawthorn extract, elderberry extract, yarrow extract, peppermint extract, mallow extract, Senburi extract (Swertia japonica extract), jujube extract, thyme extract, clove extract, Chigaya extract (Imperata grass extract), tomato extract, natto extract, wild rose extract, hibiscus extract, Bakunondou extract (Ophiopogon root extract), parsley extract, Parietaria extract, Hikiokoshi extract (Isodon leaf extract), bisabolol, coltsfoot extract, Fukinohachimitsu (coltsfoot honey), Toki extract (Japanese angelica root extract), Bukuryou extract (Poria sclerotium extract), butcher's broom extract, grape extract, propolis, loofah extract, linden extract, hop extract, pine extract, horse chestnut extract, skunk cabbage extract, Mukuroji extract (Sapindus fruit extract), peach leaf extract, cornflower extract, eucalyptus extract, apple extract, lettuce extract, milk vetch extract, rose extract, rosemary extract, and Roman chamomile extract.

Also, anti-inflammatory agents such as ε-aminocaproic acid, glycyrrhizic acid, dipotassium glycyrrhizate, β-glycyrrhetinic acid, lysozyme chloride, guaiazulene, and hydrocortisone; blood circulation promoters such as vitamin C, vitamin D, calcium pantothenate, biotin, nicotinamide, vitamin E, and vitamin E derivatives; vitamins such as β-carotene and carotenoid derivatives; active ingredients such as allantoin, diisopropylamine dichloroacetate, and 4-aminomethylcyclohexanecarboxylic acid; antioxidants such as flavonoids, tannins, lignans, and saponins; wound healing agents such as γ-oryzanol and protease; and various other optional components such as cepharanthine, capsicum tincture, hinokitiol, garlic extract iodide, pyridoxine hydrochloride, nicotinic acid, nicotinic acid derivatives, acetylpantothenyl ethyl ether, estradiol, ethinylestradiol, capronium chloride, diphenhydramine hydrochloride, camphor, nonylic acid vanillylamide, nonanoic acid vanillylamide, piroctone olamine, glyceryl pentadecanoate, l-menthol, menthol pyrrolidonecarboxylate, mononitroguaiacol, urea, resorcin, γ-aminobutyric acid, benzethonium chloride, mexiletine hydrochloride, auxin, female hormones and female hormone-like substances, cantharides tincture, cyclosporine, analgesics, tranquilizers, antihypertensive agents, antibiotics, antihistamines, and antibacterial substances can be optionally blended.

The target for administration of the external skin preparation of this embodiment is preferably a warm-blooded vertebrate, and more preferably a mammal. Examples of mammals in this specification include humans and non-human mammals such as monkeys, mice, rats, rabbits, dogs, cats, cows, horses, and pigs. The external skin preparation of this embodiment is preferably applied to a human subject who desires skin irritation improvement.

### (Manufacturing Method)

The sulfated polysaccharide constituting the active ingredient of the irritation relieving agent according to this embodiment can be manufactured from plant-based natural raw materials such as algae. The plant-based natural raw material is pulverized, dissolved by adding water. A salt that becomes a monovalent ion, such as sodium chloride, is added to the solution and heated. The sulfated polysaccharide monovalent ion salt can be separated by molecular weight separation after heating.

Here, the plant-based natural raw material means all plants existing in the natural world, including algae. Such plant-based natural raw materials, as algae, are not particularly limited, and examples include: brown algae (e.g., Laminaria (kelp), Undaria (wakame), Lessonia, Macrocystis (giant kelp), Ecklonia (kajime), Eisenia (arame), Ascophyllum, Durvillaea, etc.), red algae (e.g., Eucheuma, Gloiopeltis, Nemalion, Gelidium (agarophytes), Gracilaria (ogonori), Gloiopeltis (funori), Porphyra (nori), etc.), and green algae (e.g., Codium (mil), Ulva (sea lettuce, aosa), Enteromorpha (aonori), Caulerpa (iwazuta), Monostroma (hitoegusa), etc.). Other plants include, but are not limited to, citrus fruits, sugar beets, and apples. Among these, preferred materials include algae, particularly red algae, and typically, those using funori (Gloiopeltis) as a raw material are exemplified.

In a preferred embodiment, the method for producing an irritation relieving agent includes the steps of: dissolving Funori powder in water to prepare an aqueous Funori solution; a heating step of adding a monovalent cation to the aqueous Funori solution and heating; and fractionating the aqueous solution after heating by molecular weight fractionation to recover the sulfated polysaccharide salt. Instead of this heating step, the method may include the steps of: adding an acid (hydrochloric acid, sulfuric acid, or organic acid) to the aqueous Funori solution to make it acidic, followed by desalting; and adding a monovalent cation to the aqueous solution after desalting to neutralize it. Subsequently, the sulfated polysaccharide salt salified with a monovalent cation can be recovered by molecular weight separation.

The salt that becomes a monovalent cation used in the heating step is not particularly limited, and examples include sodium chloride, potassium chloride, sodium sulfate, and potassium sulfate. The amount of these salts added to the water in the hot water is not particularly limited, but the concentration is, for example, 0.0001 to 10 mol/L, further, for example, 0.001 to 5 mol/L, 0.01 to 2 mol/L, and preferably 0.1 to 1 mol/L.

The temperature of the hot water in the heating step is not particularly limited, but is, for example, 40° C. or higher, further, for example, 50° C. or higher, 60° C. or higher, 70° C. or higher, 80° C. or higher, 90° C. or higher, 100° C. or higher, or 120° C. or higher. Specifically, when water is used as the solvent for the hot water, it is performed at 100° C. or lower under normal pressure (1013 hPa±50 hPa), for example, at a temperature of 55 to 65° C.

The time required for this heating varies depending on the selected temperature, but is, for example, 1 hour to 2 months, for example, 1 hour to 1 month, 1 hour to 15 days, 1 hour to 10 days, 1 hour to 5 days, 1 hour to 3 days, 1 hour to 2 days, 1 hour to 1 day, 5 hours to 1 month, 5 hours to 15 days, 5 hours to 10 days, 5 hours to 5 days, 5 hours to 3 days, 5 hours to 2 days, 5 hours to 1 day, 10 hours to 1 month, 10 hours to 15 days, 10 hours to 10 days, 10 hours to 5 days, 10 hours to 3 days, or 10 hours to 2 days.

The heating step may include mixing the raw material Funori in water containing a monovalent cation as a solvent and leaving it for a certain period of time. This standing may include appropriate stirring.

The heating step is repeated one or more times, for example, 1 to 5 times. For example, it can be performed one or more times at 40 to 50° C., 50 to 60° C., 60 to 70° C., 70 to 80° C., or 50 to 70° C. Furthermore, when extraction is repeated multiple times, the temperature for each time may be different or the same. For example, a mode in which the temperature is gradually increased with each repetition is possible. Preferably, it is 50 to 60° C. or higher, and more preferably 55° C. or higher.

In the above heating step, it is also possible to use techniques such as pressurized liquid extraction (PLE), microwave assisted extraction (MAE), subcritical extraction (SE), or a combination thereof, as necessary. Subcritical extraction includes subcritical water extraction (SWE). Subcritical water extraction is also called superheated water extraction or pressurized hot water extraction (PHWE).

Subsequently, the method for recovering the sulfated polysaccharide salt can involve molecular weight fractionation of the solution after the heating step or neutralization step by a fractionation method using a molecular weight fractionation membrane or gel filtration. As a fractionation method using a molecular weight fractionation membrane, molecular weight fractionation can be performed industrially using an ultrafiltration membrane. For example, a predetermined molecular weight fraction can be prepared by using Microza (product name), which is an MF membrane (microfiltration membrane) or UF membrane (ultrafiltration membrane) manufactured by Asahi Kasei Corporation. An example of a gel filtration method is the use of Cellulofine GCL-300 manufactured by Seikagaku Corporation. Furthermore, an arbitrary molecular weight fraction can be prepared by combining these molecular weight fractionation methods and gel filtration methods.

The irritation relieving agent manufactured by the method of this embodiment can be mixed with a pharmacologically or cosmetically acceptable carrier to manufacture an external skin preparation. The external skin preparation and the pharmacologically or cosmetically acceptable carrier are as described above.

Next, the present invention will be described in more detail with reference to examples, but the present invention is not limited to these examples in any way. Note that in the following examples, the unit % of the numerical values indicating the amount of each component added means % by mass.

### EXAMPLES

### Production Example 1

Water was added to the pulverized dried Funori seaweed, and it was dissolved so as to loosen the entangled structure of the components. The dissolved part and the insoluble part were accurately separated so that they were in single-molecule units. Sodium chloride was added to the solution, and it was heated. After heating, the sodium salt of O-sulfated polysaccharide at the 6-position (hereinafter referred to as “funoran Na”) was isolated by molecular weight separation. Structural analysis of funoran Na was performed by analysis of reducing end saccharides and saccharide composition using a fluorescent labeling method with 2-aminopyridine, analysis of sulfate content using a turbidimetric method with barium chloride, and NMR analysis. It was confirmed that it had D-galactose as a saccharide constituent unit, and at least a part of this D-galactose saccharide constituent unit had a sulfate group at the 6-position. An aqueous solution containing 0.01 to 0.001% of the obtained O-sulfated polysaccharide sodium salt at the 6-position was prepared and used as a sample.

### Production Example 2

Funori (seaweed) was pulverized and dissolved by adding water. Hydrochloric acid was added to the solution to make it acidic, followed by desalting. After that, it was neutralized using sodium hydroxide. After desalting again, the sodium salt of O-sulfated polysaccharide at the 6-position (funoran Na) was able to be isolated by molecular weight separation.

### (Example 1) Irritation Relieving Test of Funoran Na

### <Subjects>

Ten Japanese women aged 20 to 60 years who self-identified as having sensitive skin and were prone to irritation were selected as participants for this test, after obtaining consent. These subjects had an average score of 2 or more in the skin irritation test using a 5% lactic acid irritant as a positive control, according to the test method below.

### <Test Method>

The skin irritation test was performed on human skin. The face was washed with warm water, and the subjects were allowed to acclimate for 20 minutes in a quiet room. A 5% lactic acid solution as a positive control (5 g of cosmetic grade lactic acid dissolved in distilled water to make 100 mL) was applied to the right cheek in an appropriate amount (approx. 20 pi), and the irritation score was judged according to the following criteria immediately after application (30 seconds later), 2 minutes after application, and 5 minutes after application. After the judgment was completed, the test area was promptly washed away with cold or warm water without rubbing.

TABLE 1

| Judgment Criteria | Rating Score |
|---|---|
| Intense irritation is felt | 4 |
| Irritation is felt | 3 |
| Slight feeling of irritation | 2 |
| No feeling at all | 1 |

The evaluation score was the average score obtained by dividing the total score immediately after application (30 seconds later), 2 minutes after application, and 5 minutes after application by the three observation times. Subjects were instructed to concentrate on the test without conversation to observe subtle irritation.

### <Irritation Relieving Test>

A test sample containing 1 ppm of funoran Na in a 5% lactic acid solution was prepared using the sample prepared in Production Example 1. Approximately 20 μl of this test sample was applied to the skin of the subjects, and the average score was taken immediately after application (30 seconds later), 2 minutes after application, and 5 minutes after application, similar to the skin irritation test. After the judgment was completed, the test area was promptly washed away with cold or warm water without rubbing.

### <Test Results>

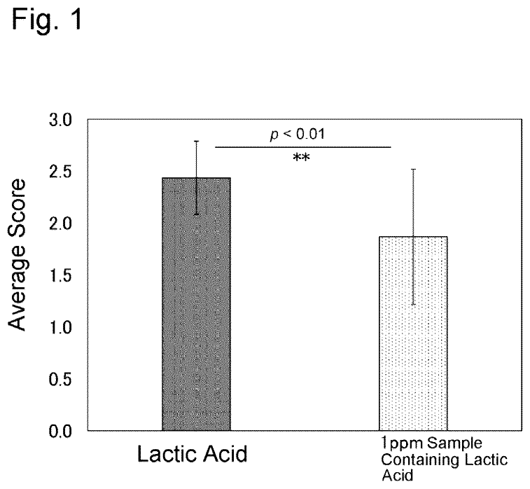

The results judged according to the same criteria as in Table 1 are shown in Table 2 below and FIG. 1.

TABLE 2

| | Lactic Acid | | | | | 1 ppm Sample Containing Lactic Acid | | | | |
|---|---|---|---|---|---|---|---|---|---|---|
| | Immediately | | | | | Immediately | | | | |
| | After | After | After | Cumulative | Average | After | After | After | Cumulative | Average |
| Subject | Application | 2 min. | 5 min. | Total | Score | Application | 2 min. | 5 min. | Total | Score |
| A | 3 | 2 | 1 | 6 | 2.0 | 2 | 1 | 1 | 4 | 1.3 |
| B | 3 | 3 | 2 | 8 | 2.7 | 2 | 1 | 1 | 4 | 1.3 |
| C | 4 | 2 | 2 | 8 | 2.7 | 4 | 3 | 2 | 9 | 3.0 |
| D | 2 | 3 | 3 | 8 | 2.7 | 1 | 2 | 2 | 5 | 1.7 |
| E | 3 | 3 | 2 | 8 | 2.7 | 2 | 2 | 1 | 5 | 1.7 |
| F | 2 | 2 | 2 | 6 | 2.0 | 2 | 2 | 2 | 6 | 2.0 |
| G | 2 | 2 | 3 | 7 | 2.3 | 1 | 1 | 2 | 4 | 1.3 |
| H | 3 | 3 | 3 | 9 | 3.0 | 4 | 3 | 2 | 9 | 3.0 |
| I | 3 | 2 | 2 | 7 | 2.3 | 3 | 2 | 1 | 6 | 2.0 |
| J | 2 | 3 | 1 | 6 | 2.0 | 2 | 1 | 1 | 4 | 1.3 |

| | | | | | |
|---|---|---|---|---|---|
| Average Value | 7.3 | 2.4 | Average Value | 5.6 | 1.9 |

| | | | |
|---|---|---|---|
| Standard Deviation (SD) | 0.4 | Standard Deviation (SD) | 0.7 |

As shown in Table 2 and FIG. 1, the average score significantly decreased when the sample containing 1 ppm of funoran Na was applied, compared to when the 5% lactic acid solution used as the positive control was applied.

### Example 2

An irritation relieving test was conducted using the same test method as in Example 1, but with a 5% lactic acid solution containing 0.1 ppm or 0.01 ppm of funoran Na (approx. 20 μl). The results are shown in Table 3 below.

TABLE 3

| | | | | | | 0.1 ppm Sample | | |
|---|---|---|---|---|---|---|---|---|
| | Lactic Acid | | | | | Containing Lactic Acid | | |
| | Immediately | | | | | Immediately | | |
| | After | After | After | Cumulative | Average | After | After | After |
| Subject | Application | 2 min. | 5 min. | Total | Score | Application | 2 min. | 5 min. |
| A | 3 | 3 | 2 | 8 | 2.7 | 3 | 3 | 2 |
| B | 2 | 2 | 2 | 6 | 2.0 | 3 | 2 | 2 |
| C | 3 | 2 | 1 | 6 | 2.0 | 3 | 1 | 1 |
| D | 3 | 3 | 2 | 8 | 2.7 | 3 | 2 | 2 |
| E | 2 | 3 | 3 | 8 | 2.7 | 3 | 4 | 4 |
| F | 3 | 3 | 3 | 9 | 3.0 | 4 | 3 | 2 |
| G | 3 | 3 | 2 | 8 | 2.7 | 3 | 2 | 2 |
| H | 3 | 2 | 2 | 7 | 2.3 | 1 | 1 | 1 |
| I | 2 | 3 | 2 | 7 | 2.3 | 2 | 2 | 1 |
| J | 2 | 2 | 3 | 7 | 2.3 | 3 | 3 | 3 |

| Average Value | | | | 7.4 | 2.5 | Average Value | | |
|---|---|---|---|---|---|---|---|---|
| Standard Deviation (SD) | | | | | 0.3 | Standard Deviation (SD) | | |
| | | 0.1 ppm Sample | | 0.01 ppm Sample Containing Lactic Acid | | | | |
| | | Containing Lactic Acid | | Immediately | | | | |
| | | Cumulative | Average | After | After | After | Cumulative | Average |
| | Subject | Total | Score | Application | 2 min. | 5 min. | Total | Score |
| | A | 8 | 2.7 | 4 | 3 | 2 | 9 | 3.0 |
| | B | 7 | 2.3 | 3 | 2 | 2 | 7 | 2.3 |
| | C | 5 | 1.7 | 2 | 1 | 1 | 4 | 1.3 |
| | D | 7 | 2.3 | 3 | 3 | 2 | 8 | 2.7 |
| | E | 11 | 3.7 | 2 | 3 | 2 | 7 | 2.3 |
| | F | 9 | 3.0 | 4 | 3 | 3 | 10 | 3.3 |
| | G | 7 | 2.3 | 3 | 2 | 1 | 6 | 2.0 |
| | H | 3 | 1.0 | 2 | 1 | 1 | 4 | 1.3 |
| | I | 5 | 1.7 | 2 | 2 | 1 | 5 | 1.7 |
| | J | 9 | 3.0 | 3 | 3 | 3 | 9 | 3.0 |

| | | | | | |
|---|---|---|---|---|---|
| Average | 7.1 | 2.4 | Average Value | 6.9 | 2.3 |
| Value | | | | | |

| | | | | |
|---|---|---|---|---|
| Standard | Standard | 0.8 | Standard Deviation (SD) | 0.7 |
| Deviation | Deviation | | | |
| (SD) | (SD) | | | |

As shown in Table 3, a tendency for the average score to decrease was observed even when 0.1 ppm or 0.01 ppm of funoran Na was added, compared to the positive control. From these skin irritation judgment results, it was found that the irritation relieving effect of funoran Na attenuated in a concentration-dependent manner below 0.1 ppm, and the certainty of the irritation relieving effect was proven at 1 ppm or higher. Therefore, it was concluded that funoran Na is effective as an irritation relieving material when blended at 0.01 to 1 ppm in substances or products that have the potential to cause skin irritation.

### INDUSTRIAL APPLICABILITY

The irritation relieving agent of the present invention has the potential to provide external skin preparations such as pharmaceuticals and cosmetics that can be freely used without discomfort by people who have previously felt skin discomfort with cosmetic products, by adding an extremely small amount to all cosmetics such as skin care products and make-up products.

1. An irritation relieving agent comprising a sulfated polysaccharide salt having a sulfate group in at least one saccharide unit structure, the sulfate group forming a salt with a monovalent cation.

2. The irritation relieving agent according to claim 1, wherein the sulfated polysaccharide salt has one or more hexose constituent units selected from the group consisting of fucose, rhamnose, galactose, glucose, mannose, and fructose, and has a sulfate group in at least one of these hexose constituent unit structures.

3. The irritation relieving agent according to claim 1, wherein the sulfated polysaccharide salt is funoran sodium salt, which is a polysaccharide derived from Funori.

4. An external skin preparation comprising the irritation relieving agent according to claim 1 in an amount of 0.000001% by mass to 0.01% by mass in terms of solid content of the sulfated polysaccharide salt.

5. A method for producing an irritation relieving agent, comprising:
dissolving Funori powder in water to prepare an aqueous Funori solution;
adding a monovalent cation to the aqueous Funori solution and heating; and
fractionating the aqueous solution after heating by molecular weight fractionation to recover the sulfated polysaccharide salt.

6. A method for producing an irritation relieving agent, comprising:
dissolving Funori powder in water to prepare an aqueous Funori solution;
adding an acid to the aqueous Funori solution to make it acidic, followed by desalting;
adding a monovalent cation to the aqueous solution after desalting to neutralize it; and fractionating the aqueous solution after neutralization by molecular weight fractionation to recover the sulfated polysaccharide salt.

7. An external skin preparation comprising the irritation relieving agent according to claim 2 in an amount of 0.000001% by mass to 0.01% by mass in terms of solid content of the sulfated polysaccharide salt.

8. An external skin preparation comprising the irritation relieving agent according to claim 3 in an amount of 0.000001% by mass to 0.01% by mass in terms of solid content of the sulfated polysaccharide salt.
